(19) United States
(12) Patent Application Publication
(10) Pub. No.: US 2003/0082109 A1
(43) Pub. Date: May 1, 2003

(54) Dextrorotary nicotine for smoking cessation

(76) Inventor: Leonard Gorenstein, Port Richey, FL (US)

Correspondence Address: SMITH & HOPEN PA, 15950 BAY VISTA DRIVE, SUITE 220, CLEARWATER, FL, 33760

(21) Appl. No.: 09/682,886

(22) Filed: Oct. 30, 2001

(51) Int. Cl. A61K 35/78; A61K 9/68; A61K 31/4439

(52) U.S. Cl. 424/48; 424/751; 514/343

(57) ABSTRACT

The present invention provides a substitute for smoking which includes, in a first embodiment, a conventional chewing gum base comprising about 40 percent, by weight, of the substitute and an amount of synthetic dextrorotary isomer of nicotine, which is only half as toxic as tobacco-derived laboratory nicotine, in a range of about 0.02 to 8.0 percent by weight relative to the chewing gum base, each piece or dosage containing an amount of the isomer of nicotine in a range of about 0.5 to 40 milligrams. A kit has a plurality of dosages covering the percent weight range of 0.02 to 8.0 and amount range of 0.5 to 40 milligrams, enabling a person to consume progressively smaller amounts of the isomer to gradually overcome an addiction to smoking. Additional embodiments include base materials other than chewing gum.

### BACKGROUND OF INVENTION

1. Field of the Invention

The present invention relates to the field of chewable or edible substitutes for smoking, and particularly for such substitutes adapted for enabling a gradual withdrawal from dependency or addiction to smoking.

2. Description of the Prior Art

Smoking tobacco products, whether in the form of cigarettes, cigars, or pipes, are detrimental and hazardous to a person's health. Numerous well-publicized studies have indicated that the incidence or risks of lung cancer, emphysema and heart problems is significantly higher in heavy smokers than in non-smokers, and that the incidence and effects increase with the extent of smoking. Many studies also indicate, if the diseases have not yet started, or some are not far advanced, that much of the risk can be eliminated by stopping or drastically curbing the smoking habit.

In spite of the apparent conclusiveness of such studies, a large percentage of the population continues to smoke. Many persons who do smoke recognize those inherent risks but feel the present satisfactions gained by smoking outweigh possible future bad effects or find that they are addicted to smoking to the extent they are unable to stop. Many who do stop smoking start smoking again, for the reasons just mentioned.

Various means and methods have been devised for trying to break a smoker's dependency or addiction to tobacco. Some of those involve ingesting of materials that cause extreme discomfort when the person later smokes. Others involve other unpleasant stimuli applied when the person smokes. The concept of such methods is to provide negative associations with smoking.

Still other methods involve substitutes for smoking. For example, when a person craves a cigarette or cigar, he instead chews a stick of gum or eats fruit. Special compounds have been disclosed to act as a substitute for tobacco. For example, see U.S. Pat. No. 107,693 to Kilbourn U.S. Pat. No. 1,474,110 to Brown.

A problem with such substitutes or unpleasant associations is that they do little or nothing to actually reduce or eliminate a smoker's addiction to tobacco. Often, this addiction is stronger than the satisfaction of the substitutes or the tobacco withdrawal symptoms are more unpleasant than the unpleasant associations.

As a result, the cessation of smoking is often only temporary; many smokers have “quit” smoking dozens of times, each time having returned to smoking to satisfy their craving for tobacco addiction. Often too, a heavy smoker undergoes undesirable personality changes while attempting to stop, and consequently is often encouraged by others to return to smoking to regain his former personality. Still others, by substituting eating for smoking rapidly gain weight upon stopping smoking and soon start smoking again to lose weight. It is thus apparent that such methods are not generally satisfactory, particularly for heavy smokers who have smoked a long time.

Other methods rearrange the addictive nature of smoking, particularly the addiction to the nicotine contained in tobacco. It appears widely accepted that the nicotine is not the most harmful agent to the smoker, but rather the tars and other products generated by the high combustion temperatures attained. Some even consider that nicotine may provide beneficial effects under controlled conditions, for example, as disclosed in U.S. Pat. No. 3,870,794 to Hutchinson et al., relating to the treatment of certain emotional disorders with nicotine compounds.

Such other methods, exemplified by the disclosures of Lichtneckert et al, U.S. Pat. Nos. 3,877,468 and 3,901,248, provide a chewable smoking substitute that contains nicotine. By chewing the substitute, a person obtains the nicotine he or she craves or is addicted to without smoking; hence, the smoking habit can purportedly be broken. In particular, U.S. Pat. No. 3,901,248 to Lichtneckert et al discloses that the substitute is made more effective by binding tobacco derived nicotine, or other nicotine-like alkaloids to a certain exchanger to cause slow release of the alkaloid. Thus, instead of being released mostly in a person“s mouth, substantial amounts of the alkaloids are released in the throat region to cause the sensation of smoking, including a burning sensation. Although it may be considered that one addiction is substituted for another, the chewable substitute is less harmful than smoking. Furthermore, it is normally intended that the use of chewable substitute be gradually phased out.

Such methods include disadvantages. Nicotine, as derived from tobacco, is one of the most toxic substances known. As such, it is even used in a variety of insecticides and pesticides. Utmost care must accordingly be exercised in using this substance, since even small excesses beyond the desired or intended amount may be harmful. Also, because of its great toxicity, only very small amounts of tobacco-derived nicotine can be used in the smoking substitutes, and providing dinnertime dosages to minimized withdrawal symptoms is very difficult and costly. Furthermore, use of some of the disclosed alternatives may be unsatisfactory. In particular, lobeline has been found and reported to have adverse side effects, which are worse than tobacco withdrawal symptoms.

As a consequence, there has heretofore been no really acceptable or effective substitute for smoking and which also enables a person to taper off and quit smoking without undergoing withdrawal symptoms.

### SUMMARY OF INVENTION

A substitute for smoking includes a base material and a preselected quantity of dextrorotary isomer of nicotine dispersed in the base material. The base material is present in an amount of about 40 percent of weight of the substitute and the isomer of nicotine is present in an amount of about 0.02 to 8.0 percent by weight relative to the weight of the base material.

The base material may be delivered to a smoker in any convenient form. For example, the base material is advantageously adapted for chewing (food items such as candies, lozenges, chewables, both metered and unmetered), sucking (food items such as candies, lozenges, etc.), eating, and may be otherwise delivered to a user by means of transdermal patches, sub-lingual sprays, both metered and non-metered, oral sprays, also both metered and unmetered, liquid concentrates, drops, or droplets, metered or unmetered, artificial tobaccos or smokeables in dispensers of any type, herbal mixtures including dextrorotary nicotine, sublingual tabs, tablets, pills, gel-caps, injections, and the like. Other methods of administration are also included even if not specifically listed herein.

Each dosage of the substitute contains an amount of the isomer in the weight range of about 0.5 to 10 milligrams. The isomer of nicotine may be bound to a cation exchanger, the weight ratio of the isomer to the exchanger being about 2:1. The base material may include a generally conventional chewing gum base if it is desired to deliver the base material in that form. The base material is otherwise adapted if it is to be delivered in a spray, through a transdermal patch, and so on as listed hereinabove.

A kit of various dosages may be provided, at least some of the dosages containing different amounts of the isomers of nicotine. For example, some dosages may contain the dextrorotary isomer of nicotine in the range of 16 to 20 milligrams, others in the range of 12 to 16 milligrams, others in the range of 8 to 12 milligrams, and so forth. By use of the dosages having decreasing amounts of the isomer, a user may gradually withdraw from the smoking addiction. The different dosages are coded in a manner enabling identification of at least the relative amounts of the isomer of nicotine present.

Because the dextrorotary isomer of nicotine is only half as toxic as the tobacco-derived laboratory isomer of nicotine used in heretofore available smoking substitutes, more of the dextrorotary isomer may be used to provide greater user satisfaction at the same level of toxicity, or less toxicity at the same level of satisfaction. By enabling use of greater amounts of nicotine, by using the dextrorotary isomer, a wide range of dosages can be provided to enable more gradual tapering off of smoking dependency.

### DETAILED DESCRIPTION

Nicotine is a colorless, oily liquid having the chemical formula of C10H14N2, formally referred to as 1 methyl-2-(3-Pyridyl) pyrrolidine and is present in tobacco to the extent of 1.8 percent. Nicotine is also a molecular asymmetrical organic compound; as such, it exhibits optical activity, i.e., nicotine has the property of causing rotation of plane polarized light passed therethrough.

As derived from tobacco, nicotine is a levoratory (l-) compound, causing plane polarized light passed therethrough to rotate to the left. Nicotine may, however, also be chemically synthesized in a manner known to those skilled in the art, and from the synthesized compound, a dextrorotary (d-) nicotine isomer or compound may be isolated.

Both the tobacco derived l-compound or isomer and the synthesized d-compound or isomer have identical chemical formulas (C10H14N2) have the same chemical reactions and are alike in physical properties. However, they differ in physiological action and effect. Stated otherwise, the l- and d-isomers have different effects on living organisms. Accordingly, it has been found and reported that the synthesized d-isomer of nicotine is only one half as toxic as the natural tobacco-derived l-isomer.

This difference in toxicity of the l- and d-isomers is very important in formulating smoking substitutes and in formulating them in a manner that they can be used to gradually diminish and eliminate a person's dependence on smoking, particularly since both isomers otherwise give the same effect and sensations, to the same degree, to a person. Thus, a given amount of the synthetic d-isomer of nicotine gives a person the same sensations and satisfaction as a like amount of the natural, l-isomer nicotine, but at only half the toxicity, or at the same level of toxicity, the d-isomer provides greater effects and satisfaction to a user than does the l-isomer.

Several results follow from the reduced toxicity of the d-isomer. More of the d-isomer nicotine can safely be used in a smoking substitute such as chewing gum, candy, lozenges, tablets, etc., than of the l-isomer nicotine; consequently, a substitute having greater user satisfaction can be provided with no greater, or even less, toxic reaction. Secondly, because a greater amount of the d-isomer nicotine can be used in the smoking substitute, normal tolerances in compounding the substitute have relatively less effect. Thirdly, because the d-isomer nicotine can be used in the smoking substitute, without increasing toxicity, it is possible to more easily and practically formulate a graduated series of dosages having decreasing amounts of nicotine, so that over a period of time a person can be gradually weaned away from smoking without experiencing withdrawal symptoms.

Accordingly, whereas previous smoking substitutes, for example, chewing gum have been disclosed in which the percentage of weight of l-isomer nicotine relative to the base material used varies from about 0.05 percent to 2.0 percent, the percentage weight of the d-isomer nicotine preferably varies from about 0.02 percent to 8.0 percent. Whereas the weight per dosage of the l-isomer has been disclosed to vary from about 1 to 10 milligrams, the weight per dosage of the d-isomer may vary from about 0.5 to 40 milligrams.

Use is possible of a wide variety of known chewing gum bases, with the d-isomer of nicotine, the only requirement being that a relatively high gum base concentration be used. Such concentration should be at least about 40 percent by weight of the chewing gum as ready for use. Examples of the gum bases that may be used to advantage are Chicle-, Percha-, Malaya- and Jelutong-bases. Natural resins, such as Dammar and Mastix may also be used, as may synthetic gum bases such as polyvinylacetate polyvinylesters and polyisobutylene. Again, as mentioned above, the inventive base may be delivered by many means other than as a chewing gum base.

Softeners, such as are normally added to a chewing gum base to improve texture, to reduce viscosity, etc. in conventional chewing gums are preferably also added to the chewing gum described herein. Examples of such commonly used softeners are lanolin, hydrogenated cotton seed or coconut oil, mineral oil, glycerine, honey and olive oil.

Other commonly used additives, such as starch, calcium carbonate, talcum, defatted cocoa, flavoring and colorings may also be used. Sugar, in the form, for example, of sucrose or corn syrup may also be added. Sugar-free chewing gum may also be used in conjunction with the d-isomer nicotine.

Preferably, to delay release of all of the d-isomer nicotine, the isomer is bound to a cation exchanger of the type described in U.S. Pat. No. 3,901,248 to Lichtneckert et al. By binding the isomer to a cation exchanger, absorption of the isomer in the mouth is reduced and more of the isomer is carried to throat regions, to provide more of the sensations associated with smoking, since for optimum effectiveness the smoking substitute should closely approximate those associated with smoking.

Actual formulation of chewing gum, exclusive of the d-isomer of nicotine and the cation exchanger, to which it may be bound if desired, is well known to those in the chewing gum art and consequently need not be described herein in detail, it being understood that the d-isomer, as well as the cation exchanger, can be used with substantially any commercial chewing gum process, whether or not sugar-free.

The smoking substitute chewing gum, according to the present invention can be made most conveniently by mixing a conventionally compounded chewing gum base or mass with the d-isomer or with the d-isomer cation exchanger, the latter preferably also including an excess of a suitable acidifying agent. In either case, the d-isomer or d-isomer exchanger should be thoroughly and intimately mixed with the chewing gum mass, somewhat elevated temperatures being used, if necessary. The d-isomer or d-isomer exchanger are conveniently added to the chewing gum mass along with the addition of other conventional additives such as sugars or flavorings. Depending on the properties of the acidifying agent, if one is to be used, it will be found convenient, for example to add liquid acidifying agents, for example, sulphuric acid, with conventional liquid additives such as corn syrup. Non-liquid acidifying agents, for example, malic acid, may be added with conventional non-liquid additives, such as powdered sugar.

Preferably, also, the d-isomer or d-isomer exchanger is added when the chewing gum mass has cooled to an extent that the mass is just mixable, in order to prevent losses of the isomer which may occur at high temperatures.

After such additives and mixing, the chewing gum is formed into sticks, or pieces in a conventional manner. The weight of each stick or piece is preferably that of conventional sticks or pieces, i.e., between about 1.0 to 3.0 grams. The weight percentage of the d-isomer, compared to the weight of the chewing gum base is preferably in the range of about 0.02 to 8.0 percent, the weight of the d-isomer in each stick or piece being in the range of about 0.5 to 10 milligrams. When a cation exchanger, for example of the types disclosed in U.S. Pat. No. 3,901,248 to Lichtneckert et al. is used, the ratio of d-isomer to exchanger is preferably about 2:1, instead of the 1:1 ratio disclosed for the nicotine type used.

An effective manner to enable withdrawal of a person from dependency upon, or addiction to, smoking is by forming the chewing gum, lozenge, tablet, etc., in such a manner that some sticks, or pieces, etc., have a relatively high concentration of the d-isomer and others have a lower concentration. Thus a kit or package is provided in which some sticks or pieces of gum, lozenges, etc. have, e.g., about 16 to 20 milligrams of the d-isomer, others have, e.g., about 12 to 16 milligrams, others about 8 to 12 milligrams and so forth until some have only 1 to 4 milligrams. The sticks or pieces are color, size, shape, or otherwise coded in a manner enabling ready identification of at least the relative amounts of the d-isomer present. For example, sticks or pellets having about 20 milligrams of the d-isomer may be colored red, those having about 16 milligrams may be colored orange, and so forth.

When use of the substitute is started, and smoking is stopped, sticks or pieces of chewing gum, lozenges, etc. having the highest amount of the d-isomer are chewed, or sucked, or sprayed, etc, e.g., for several days or a week. Then, over a period of time, sticks or pieces with gradually decreasing amounts of the d-isomer are used, the user being gradually withdrawn from the dependency upon smoking without experiencing withdrawal symptoms.

It is to be appreciated that the smoking substitute is not limited to chewing gum, although that is a useful form since the d-isomer nicotine is gradually released in a person“s mouth. Other forms, which may be used to advantage, are lozenges, which a person sucks, or a food item such as candy that is slowly eaten. The use of transdermal patches, sub-lingual sprays, both metered and unmetered, oral sprays, also both metered and unmetered, liquid concentrates, drops, or droplets, metered or unmetered, artificial tobaccos or smokeables in dispensers of any type, herbal mixtures including dextrorotary nicotine, sublingual tabs, tablets, pills, gel-caps, injections, and the like, are all within the scope of this invention. Other methods of administration are also included even if not specifically listed herein.

With such alternatives, the d-isomer or d-isomer cation exchanger is added as generally described above for chewing gum, to the other materials as they are being conventionally compounded. Graduated amounts of the d-isomer may be provided in coded ones of these alternatives, again as described above for the chewing gum form.

Although there has been described above a chewable substitute for smoking and a substitute for smoking kit, in accordance with the invention for the purpose of illustrating the manner in which the invention may be used to advantage, it will be appreciated that the invention is not limited thereto. Accordingly, any and all modifications, variations or equivalent arrangements which may occur to those skilled in the art should be considered to be within the scope of the invention as defined in the appended claims.

Now that the invention has been described,

1. A smoking substitute, comprising:
a base material comprising about 40 percent by weight of a dosage of the substitute, said base material being adapted for delivery to a user; and
a preselected quantity of dextrorotary isomer of nicotine uniformly dispersed in the base material and being present in an amount in the range of about 0.02 to 8.0 percent by weight compared to the weight of the base material, the amount of said isomer of nicotine dispersed in the base material being in the range of about 0.5 to 40 milligrams per dosage of the smoking substitute.

2. The smoking substitute of claim 1, wherein the base material is adapted for delivery to a user in a chewing gum base.

3. The smoking substitute of claim 1, wherein the base material is adapted for delivery to a user over a predetermined period of time by a transdermal patch.

4. The smoking substitute of claim 1, wherein the base material is adapted for delivery to a user by an oral spray.

5. The smoking substitute of claim 1, wherein the base material is adapted for delivery to a user by a food item.

6. The smoking substitute of claim 1, wherein the base material is adapted for delivery to a user by a lozenge.

7. The smoking substitute of claim 1, wherein the base material is adapted for delivery to a user by a liquid concentrate.

8. The smoking substitute of claim 1, wherein the base material is adapted for delivery to a user by a smokeable substance such as artificial tobacco.

9. The smoking substitute of claim 1, wherein the base material is adapted for delivery to a user by an herbal mixture.

10. The smoking substitute of claim 1, wherein the base material is adapted for delivery to a user by sublingual tabs.

11. The smoking substitute of claim 1, wherein the base material is adapted for delivery to a user by tablets, pills, and gel caps.

12. The smoking substitute of claim 1, wherein the base material is adapted for delivery to a user by injection.

13. The smoking substitute of claim 1, further comprising a cation exchanger to which said isomer of nicotine is bound.

14. The smoking substitute of claim 12, wherein the isomer of nicotine and cation exchanger are present in the weight ratio of about 1:2.

15. The smoking substitute of claim 1, including means for providing the smoking substitute in graduated dosage kit form, enabling a person to be gradually withdrawn from dependency on smoking, said means including a plurality of dosages of the smoking substitute, each dosage including different amounts of said isomer of nicotine dispersed in the base material, said different amounts being sub ranges in said range of about 0.5 to 10 milligrams.

16. The smoking substitute of claim 14, wherein said sub ranges include ranges of about 1 to 4 milligrams, about 4 to 8 milligrams, about 8 to 12 milligrams, about 12 to 16 milligrams and about 16 to 20 milligrams.

17. The smoking substitute of claim 14, wherein said plurality of dosages having different amounts of said isomer of nicotine includes means for identifying at least the relative amounts of said isomer of nicotine in each of said dosages.

18. A substitute smoking kit, comprising a plurality of dosages of a smoking substitute, each of said dosages including a base material comprising about 40 percent by weight of the substitute and being of a chewable, suckable or edible variety, and a preselected quantity of dextrorotary isomer of nicotine uniformly distributed in said base material, said plurality of dosages covering a range of about 0.02 to 8.0 percent by weight of said isomer relative to said base material, and correspondingly covering an amount of said isomer per dosage in the range of 0.5 to 40 milligrams, at least some of said plurality of dosages containing substantially different amounts of said isomer within said 0.5 to 40 milligram range.

19. The substitute smoking kit of claim 17, including means for distinguishing dosages having different amounts of said isomer, whereby a person can select desired ones of the dosages.

20. The substitute smoking kit of claim 17, wherein each of said dosages also includes a cation exchanger to which the isomer of nicotine is bound.
